(19) United States
(12) Patent Application Publication
(10) Pub. No.: US 2026/0116767 A1
(43) Pub. Date: Apr. 30, 2026

(54) SMALL CRYSTAL SBS/SBT ZEOLITE INTERGROWTH, ITS METHOD OF MAKING AND USE

(71) Applicant: CHEVRON U.S.A. INC., San Ramon, CA (US)

(72) Inventors: Taekyung RYU, Richmond, CA (US); Howard Steven LACHEEN, Richmond, CA (US); Joel Edward SCHMIDT, Oakland, CA (US); Marat Borisovich ORAZOV, El Cerrito, CA (US)

(21) Appl. No.: 19/373,061

(22) Filed: Oct. 29, 2025

Related U.S. Application Data
(60) Provisional application No. 63/713,467, filed on Oct. 29, 2024.

(51) Int. Cl. C01B 39/48; B01J 29/74; B01J 37/08; B01J 37/30; C01B 39/02; C07C 5/22

(52) U.S. Cl. CPC C01B 39/48; B01J 29/74; B01J 37/088; B01J 37/30; C01B 39/026; C07C 5/22; C01P 2002/60; C01P 2002/72; C01P 2004/03

(57) ABSTRACT

Provided herein is a small crystal size aluminosilicate zeolite having an intergrowth structure of a SBS framework structure and a SBT framework structure, designated as SSZ-133. The aluminosilicate zeolite may be synthesized by (a) preparing a synthesis mixture comprising a source of alumina, a source of silica, an organic template containing a tetramethylammonium cation, a source of hydroxide ions, a source of cesium cation, and water; (b) heating the synthesis mixture under crystallization conditions including a temperature of from 50° C. to 150° C. for a time sufficient to form crystals of the zeolite; and (c) recovering at least a portion of the zeolite from step (b). The source of alumina is free of, essentially free of, or substantially free of an aluminum alkoxide and the source of silica is free of, essentially free of, or substantially free of colloidal silica.

### RELATED APPLICATIONS

This application claims priority to and the benefit of U.S. Provisional Application No. 63/713,467, filed Oct. 29, 2024, the disclosure of which is incorporated herein by reference in its entirety.

### TECHNICAL FIELD

The present disclosure relates to small crystal size SBS/SBT zeolite intergrowth compositions, designated as SSZ-133, methods of making the same, and uses thereof.

### BACKGROUND

Molecular sieve materials, both natural and synthetic, may be used as adsorbents and have catalytic properties for hydrocarbon conversion reactions. Certain molecular sieves, such as zeolites, are porous crystalline materials which have an ordered structure as determined by X-ray diffraction. Within such materials there are a large number of uniform cavities and pores which may be interconnected by a number of channels. The sizes and dimensions of these cavities and pores are uniform within a specific molecular sieve material and allow for adsorption of molecules of certain sizes while rejecting those of larger dimensions. Due to their ability to adsorb molecules through size selections, molecular sieves and zeolites have many uses including hydrocarbon conversion processes, e.g., cracking, hydrocracking, disproportionation, alkylation, oligomerization, and isomerization. Molecular sieves that find application in catalysis and adsorption include any of the naturally occurring or synthetic crystalline molecular sieves. Examples of these molecular sieves include extra-large pore zeolite, large pore zeolites, medium pore size zeolites, and small pore zeolites. These zeolites and their isotypes are classified by the Structure Commission of the International Zeolite Association according to the rules of the IUPAC Commission on Zeolite Nomenclature. According to this classification, framework type zeolites and other crystalline microporous molecular sieves, for which a structure has been established, are assigned a three-letter code and are described in the “Atlas of Zeolite Framework Types”, eds. Ch. Baerlocher, L. B. McCusker, and D. H. Olson, Elsevier, Sixth Revised Edition, 2007, which is hereby incorporated by reference. These zeolites and their isotypes are also described in the database of zeolite structures, which provides structural information on all of the zeolite framework types that have been approved by the Structure Commission of the International Zeolite Association (IZA-SC).

The idealized inorganic framework structure of zeolites is a framework of silicate in which all tetrahedral atoms are connected by oxygen atoms with the four next-nearest tetrahedral atoms (T-atoms). The term “silicate”, as used herein, refers to a substance containing at least silicon and oxygen atoms that are alternately bonded to each other (i.e., —O—Si—O—Si—), and optionally including other atoms within the inorganic framework structure, including atoms such as boron, aluminum, or other metals (e.g., transition metals, such as titanium, iron, or zinc). Atoms other than silicon and oxygen in the framework silicate occupy a portion of the lattice sites otherwise occupied by silicon atoms in an ‘all-silica’ framework silicate. Thus, the term “framework silicate” as used herein refers to an atomic lattice comprising any of a silicate, borosilicate, aluminosilicate, titanosilicate, ferrisilicate, zincosilicate, or the like.

The framework silicates of zeolites or molecular sieves are commonly characterized in terms of their ring size, wherein the ring size refers to the number of silicon atoms (or alternative atoms, such as those listed above) that are tetrahedrally coordinated with oxygen atoms in a loop to define a pore or channel within the interior of the zeolite. For example, a “12-ring” zeolite is a zeolite having pores or channels defined by 12 alternating tetrahedral atoms and 12 oxygen atoms in a loop. The pores or channels defined within a given zeolite may be symmetrical or asymmetrical depending upon various structural constraints that are present in the particular framework silicate.

Zeolites can be classified as having small, medium, large, and extra-large pore structures for pore windows delimited by 8, 10, 12, and more than 12 T-atoms, respectively. Extra-large pore zeolites (>12R) include, for example, AET (14R, e.g., ALPO-8), SFN (14R, e.g., SSZ-59), VFI (18R, e.g., VPI-5), CLO (20R, e.g., cloverite), and ITV (30R, e.g., ITQ-37) framework type zeolites. Extra-large pore zeolites generally have a free pore diameter of larger than about 0.8 nm. Large pore zeolites (12R) include, for example, LTL, MAZ, FAU, EMT, OFF, *BEA, and MOR framework type zeolites, e.g., zeolite L, mazzite, omega, zeolite Y, zeolite X, zeolite Y, ZSM-2, zeolite T, offretite, and Beta. Large pore zeolites generally have a free pore diameter of 0.6 to 0.8 nm. Medium (or intermediate) pore size zeolites (10R) include, for example, MFI, MEL, EUO, MTT, MFS, AEL, AFO, HEU, FER, MWW, and TON framework type zeolites, e.g., ZSM-5, ZSM-11, ZSM-22, MCM-22, silicalite-1, and silicalite-2. Medium pore size zeolites generally have a free pore diameter of 0.45 to 0.6 nm. Small pore size zeolites (8R) include, for example, CHA, RTH, ERI, KFI, LEV, and LTA framework type zeolites, e.g., ZK-4, SAPO-34, SAPO-35, ZK-14, SAPO-42, ZK-21, ZK-22, ZK-5, ZK-20, zeolite A, chabazite, and ALPO-17. Small pore size zeolites generally have a free pore diameter of 0.3 to 0.45 nm.

Molecular sieves may have ordered or disordered structure. Ordered molecular sieves are ordered in three dimensions. When the crystal structure is ordered in all three dimensions, the structure is called an ordered end member structure. Disordered structures (intergrowths), on the other hand, show periodic ordering in less than three dimensions. Such intergrowths frequently have significantly different catalytic properties from their end members.

PST-2 is a synthetic aluminosilicate zeolite having an intergrowth structure of cage-based, large pore SBS and SBT framework structures. Multi-dimensional large pore zeolites with supercages, such as PST-2, are of significant interest as potential catalysts, particularly for refining and petrochemical processes that conventionally utilize zeolite Y based catalysts.

As conventionally synthesized, for example as taught by Korean Patent No. 10-2546689, a typical preparation of PST-2 produces large crystals of greater than 1 mm in size. Such large crystals inherently have slower diffusion. For some acid-catalyzed reactions over zeolites, it is beneficial to reduce diffusion lengths of the reagent and/or product molecules by employing a zeolite with a reduced crystal size. Therefore, a need exists for small crystal forms of SBS/SBT zeolite intergrowth materials.

### SUMMARY

The present disclosure relates to small crystal size SBS/SBT zeolite intergrowth compositions, methods of making the same, and uses thereof.

In a first aspect, the present disclosure relates to an aluminosilicate zeolite having an intergrowth structure of a SBS framework structure and a SBT framework structure, wherein the zeolite has a d50 crystal size of 500 μm or less.

In a second aspect, the present disclosure relates to method of making an aluminosilicate zeolite, in particular a zeolite as defined in the first aspect of the present disclosure, comprising: (a) preparing a synthesis mixture comprising a source of alumina, a source of silica, an organic template comprising a tetraethylammonium cation, a source of hydroxide ions, a source of cesium cation, and water; (b) heating the synthesis mixture under crystallization conditions including a temperature of from 50° C. to 150° C. for a time sufficient to form crystals of the zeolite; and (c) recovering at least a portion of the zeolite from step (b); wherein the source of alumina is free of, essentially free of, or substantially free of an aluminum alkoxide and wherein the source of silica is free of, essentially free of, or substantially free of colloidal silica.

In a third aspect, the present disclosure relates to a process of converting an organic compound to a conversion product comprises contacting the organic compound with an aluminosilicate zeolite according to the first aspect of the present disclosure or prepared according to the method of the second aspect of the present disclosure.

### BRIEF DESCRIPTION OF THE DRAWINGS

FIG. 1 shows the powder X-ray diffraction (XRD) pattern of the as-synthesized product of Example 2.

FIGS. 2A and 2B show Scanning Electron Microscopy (SEM) images of the as-synthesized PST-2 zeolite product of Example 1 and the as-synthesized product of Example 2, respectively.

FIG. 3 shows the powder XRD pattern of the as-synthesized product of Example 3.

FIGS. 4A and 4B show SEM images of the as-synthesized PST-2 zeolite product of Example 1 and the as-synthesized product of Example 3, respectively

FIG. 5 shows the powder XRD pattern of the as-synthesized product of Example 4.

FIG. 6 shows a SEM image of the as-synthesized product of Example 4.

FIG. 7 shows the powder XRD pattern of the as-synthesized product of Example 5.

FIGS. 8A and 8B show SEM images of the as-synthesized PST-2 zeolite product of Example 1 and the as-synthesized product of Example 5, respectively.

FIG. 9 shows the powder XRD pattern of the as-synthesized product of Example 6.

FIG. 10 shows a SEM image of the as-synthesized product of Example 6.

FIG. 11 shows a plot of conversion or yield versus temperature for n-hexadecane (n-C16) conversion over a Pd/SSZ-133 catalyst prepared using the SSZ-133 zeolite of Example 2.

FIG. 12 shows a plot of isomer distribution versus temperature for n-C16 conversion over the Pd/SSZ-133 catalyst prepared using the SSZ-133 zeolite of Example 2.

FIG. 13 shows a plot of conversion or yield versus temperature for n-C16 conversion over a Pd/SSZ-133 catalyst prepared using the SSZ-133 zeolite of Example 3.

FIG. 14 shows a plot of isomer distribution versus temperature for n-C16 conversion over the Pd/SSZ-133 catalyst prepared using the SSZ-133 zeolite of Example 3.

FIG. 15 shows a plot of conversion or yield versus temperature for n-C16 conversion over a Pd/SSZ-133 catalyst prepared using the SSZ-133 zeolite of Example 4.

FIG. 16 shows a plot of isomer distribution versus temperature for n-C16 conversion over the Pd/SSZ-133 catalyst prepared using the SSZ-133 zeolite of Example 4.

FIG. 17 shows a plot of conversion or yield versus temperature for n-C16 conversion over a Pd/SSZ-133 catalyst prepared using the SSZ-133 zeolite of Example 5.

FIG. 18 shows a plot of isomer distribution versus temperature for n-C16 conversion over the Pd/SSZ-133 catalyst prepared using the SSZ-133 zeolite of Example 5.

FIG. 19 shows a plot of conversion or yield versus temperature for n-C16 conversion over a Pd/SSZ-133 catalyst prepared using the SSZ-133 zeolite of Example 6.

FIG. 20 shows a plot of isomer distribution versus temperature for n-C16 conversion over the Pd/SSZ-133 catalyst prepared using the SSZ-133 zeolite of Example 6.

### DETAILED DESCRIPTION

The present disclosure relates to small crystal size SBS/SBT zeolite intergrowth compositions, methods of making the same, and uses thereof. The small crystal size SBS/SBT zeolite intergrowth compositions may be designated as SSZ-133 molecular sieves, or SSZ-133 zeolites, or SSZ-133 materials.

As used herein, the term “zeolite” refers to microporous crystalline aluminosilicates composed of TO4 tetrahedra (T=Si, Al) with O atoms connecting neighboring tetrahedra.

As used herein, the term “SBS” refers to the SBS type topology or framework structure as recognized by the Structure Commission of the International Zeolite Association. The SBS framework structure is a large pore zeolite structure belonging to the space group of a hexagonal crystal system P63/mmc based on a cage in which the opening or pores is formed by a ring of 12 oxygen atoms. Crystal axis lengths a and b of the SBS framework structure may be 17.0 to 18.5 Å, specifically 17.0 to 18.0 Å, and most specifically 17.0 to 17.5 Å, respectively, but are not limited thereto. In addition, a crystal axis unit cell length c of the SBS framework structure may be 25.0 to 30.0 Å, specifically 26.0 to 28.0 Å, but is not limited thereto.

As used herein, the term “SBT” refers to the SBT type topology or framework structure as recognized by the Structure Commission of the International Zeolite Association. The SBT framework structure is a large pore zeolite structure belonging to a space group of a trigonal crystal system R-3m based on a cage in which the pore opening is formed of a ring of 12 oxygen atoms. Crystal axis unit cell lengths a and b of the SBT framework structure may be 17.0 to 18.5 Å, specifically 17.0 to 18.0 Å, and most specifically 17.0 to 17.5 Å, respectively, but are not limited thereto. In addition, a crystal axis unit cell length c of the SBT framework structure may be 41.0 to 42.5 Å, specifically 41.0 to 42.0 Å, but is not limited thereto.

As used herein, the term “intergrowth” refers to a zeolite having a framework comprising at least two different zeolite framework structures. An intergrowth is not a simple mixture of zeolites having different framework structures.

As used herein, the term “substantially free” refers to the presence of no more than 0.05%, preferably no more than 0.01%, and more preferably no more than 0.001%, of an indicated material in a composition, by total weight of such composition.

As used herein, the term “essentially free” means that the indicated material is not deliberately added to the composition, or preferably not present at analytically detectable levels. It is meant to include compositions whereby the indicated material is present only as an impurity of one of the other materials deliberately added.

Values, ranges, or features may be expressed herein as “about”, from “about” one particular value, and/or to “about” another particular value. When such values, or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means ±20% of the stated value, ±15% of the stated value, ±10% of the stated value, ±5% of the stated value, ±3% of the stated value, or ±1% of the stated value.

In a first aspect, the present disclosure relates to an aluminosilicate zeolite having an intergrowth of a SBS framework structure and a SBT framework structure, wherein the zeolite has a d50 crystal size of 500 μm or less.

In one or more embodiments, the aluminosilicate zeolite crystals have a d50 crystal size that is 500 nm or less, for example, about 250 nm or less, about 100 nm or less, from about 25 nm or 50 nm up to about 500 nm, from about 25 nm or 50 nm up to about 100 nm, or from about 100 nm to about 250 nm. In one or more embodiments, the aluminosilicate zeolite crystals have a d90 crystal size that is 500 nm or less, for example, about 250 nm or less, about 100 nm or less, from about 25 nm or 50 nm up to about 500 nm, from about 25 nm or 50 nm up to about 100 nm, or from about 100 to about 250 nm. The aluminosilicate zeolite crystals may have both a d50 and a d90 value as described above.

The crystal size is based on individual crystals. Crystal size is the length of longest diagonal of the three-dimensional crystal. Direct measurement of the crystal size can be performed using microscopy methods, such as SEM and TEM. For example, measurement by SEM involves examining the morphology of materials at high magnifications (typically 1000× to 100,000×). The SEM method can be performed by distributing a representative portion of the zeolite powder on a suitable mount such that individual particles are reasonably evenly spread out across the field of view at 1000× to 100,000× magnification. From this population, a statistically significant sample of random individual crystals (e.g., 50-200) are examined and the longest diagonal of the individual crystals are measured and recorded. Particles that are clearly large polycrystalline aggregates should not be included the measurements. Based on these measurements, the d50 and d90 of the sample crystal sizes are calculated.

In one or more further embodiments, the aluminosilicate zeolite includes crystals wherein the overall ratio of SBS framework structure to SBT framework structure is from about 5:95 to about 95:5, preferably about 60:40 to 40:60. In an exemplary embodiment, the mole ratio of SBS:SBT may range from about 5:95 to about 95:5. Examples of other framework structure ratios include about 10:90, about 15:85, about 20:80, about 25:75, about 30:70, about 40:60, about 50:50, about 60:40, about 70:30, about 75:25, about 80:20, about 85:15, and about 90:10 SBS:SBT. The mole ratio can be determined by analytical techniques such as X-ray diffraction analysis. In particular, the mole ratio can be determined by DIFFaX analysis of the X-ray diffraction patterns collected on a dehydrated H-form for a given sample.

In one or more further embodiments, the aluminosilicate zeolite has a SiO₂/Al₂O₃ molar ratio (SAR) of from 1 to 65, such as at least 2, at least 3, at least 4, or at least 5, and up to 50, or up to 25, or up to 10, e.g., 5 to 10.

In one or more further embodiments, the aluminosilicate zeolite, in its as-calcined form, may have a micropore volume of 0.10 to 0.40 cm³/g, or from 0.20 to 0.40 cm³/g, or from 0.22 to 0.38 cm³/g.

In a second aspect, the present disclosure relates to a method of making an aluminosilicate zeolite, in particular a zeolite as defined in the first aspect of the present disclosure, comprising the following steps: (a) preparing a synthesis mixture comprising a source of alumina, a source of silica, an organic template comprising a tetraethylammonium cation, a source of hydroxide ions, a source of cesium cation, and water; (b) heating the synthesis mixture under crystallization conditions including a temperature of from 50° C. to 150° C. for a time sufficient to form crystals of the zeolite; and (c) recovering at least a portion of the zeolite from step (b); wherein the source of alumina is free of, essentially free of, or substantially free of an aluminum alkoxide and wherein the source of silica is free of, essentially free of, or substantially free of colloidal silica.

The synthesis mixture can be prepared according to conventional methods. The components of the synthesis mixture may be combined in any order.

The synthesis mixture comprises a source of alumina (Al₂O₃). In one or more embodiments, the source of alumina is free of, essentially free of, or substantially free of an aluminum alkoxide (e.g., aluminum tri-sec-butoxide). Suitable sources of alumina herein may include aluminum salts, especially water-soluble salts, such as aluminum sulfate, aluminum nitrate, aluminum hydroxide, alkali metal aluminates such as sodium aluminate and potassium aluminate, as well as hydrated aluminum oxides, such as boehmite, gibbsite, and pseudoboehmite, and mixtures thereof. In one or more embodiments, the source of alumina may comprise aluminum hydroxide, or consist essentially of aluminum hydroxide, or consist of aluminum hydroxide.

The synthesis mixture comprises a source of silica (SiO₂). In one or more embodiments, the source of silica is free of, essentially free of, or substantially free of colloidal silica (i.e., aqueous colloidal suspensions of silica). Suitable sources of silica herein may include silicon alkoxides (e.g., tetramethyl orthosilicate, tetraethyl orthosilicate), fumed silica, precipitated silica, and alkali metal silicates such as potassium silicate and sodium silicate. In one or more embodiments, the source of silica may comprise fumed silica, or consist essentially of fumed silica, or consist of fumed silica.

The synthesis mixture may have a SiO₂/Al₂O₃ molar ratio (SAR) of from 1 to 70, such as from 5 to 70, or from 5 to 60, or from 5 to 50, or from 5 to 40, or from 5 to 30, or from 5 to 20, or from 10 to 70, or from 10 to 60, or from 10 to 50, or from 10 to 40, or from 10 to 30, or from 10 to 20.

The organic template (Q) comprises a tetraethylammonium cation. The organic template (Q) may be present in any suitable form, for example as a halide, such as an iodide or a bromide, or as a hydroxide, for instance in its hydroxide form. The organic template (Q) may be present in the synthesis mixture in a Q/SiO₂ molar ratio of from 0.5 to 1.5, such as from 0.7 to 1.3, from 0.9 to 1.1, or about 1:1. The organic template (i.e., tetraethylammonium cation) can be used alone or in combination with one or more other organic templates. For example, the organic template may comprise a tetraethylammonium cation and a tetramethylammonium cation in a desired molar ratio. In embodiments where the organic template comprises a tetraethylammonium cation and a tetramethylammonium cation, the molar ratio of tetraethylammonium cation to tetramethylammonium cation can range from 1:1 to 10:1, for example from 1:1 to 5:1.

The synthesis mixture contains at least one source of hydroxide ions (OH). For example, hydroxide ions can be present as a counter ion of the organic template (Q) or by the use of aluminum hydroxide as a source of aluminum. Suitable sources of hydroxide ions can also be selected from the group consisting of alkali metal hydroxides, alkaline earth metal hydroxides, ammonium hydroxide, and mixtures thereof; such as from sodium hydroxide, potassium hydroxide, lithium hydroxide, rubidium hydroxide, cesium hydroxide, calcium hydroxide, magnesium hydroxide, ammonium hydroxide, and mixtures thereof. The synthesis mixture may comprise the hydroxide ions source in a OH/SiO₂ molar ratio of from 0.75 to 1.75, such as from 0.75 to 1.5, from 1.00 to 1.75, or from 1.00 to 1.50.

The synthesis mixture comprises at least one source of cesium cation (Cs). The cesium source may be cesium hydroxide, cesium formate, cesium acetate, cesium citrate, cesium chloride, cesium bromide, cesium nitrate, or cesium sulfate. The synthesis mixture comprises the cesium cation (Cs) source in a Cs/SiO₂ molar ratio of from 0.010 to 0.15, such as from 0.010 to 0.10, or from 0.010 to 0.075, or from 0.015 to 0.15, or from 0.015 to 0.10, or from 0.015 to 0.075.

The synthesis mixture typically comprises water in a H₂O/SiO₂ molar ratio of from 10 to 60, such as from 20 to 40. Depending on the nature of the components in the base mixture, the amount of solvent (e.g., water from the hydroxide solution, and optionally methanol and ethanol from the hydrolysis of silica sources) of the base mixture may be removed such that a desired solvent to SiO₂ molar ratio is achieved for the synthesis mixture. Suitable methods for reducing the solvent content may include evaporation under a static or flowing atmosphere such as ambient air, dry nitrogen, dry air, or by spray drying or freeze drying. Water may be added to the resulting mixture to achieve a desired H₂O/SiO₂ molar ratio when too much water is removed during the solvent removal process. In some examples, water removal is not necessary when the preparation has sufficient H₂O/SiO₂ molar ratio. In the context of the present disclosure, where the term “water” is used, this term preferably describes water having a conductivity of at most 50 μS/cm.

The synthesis may be performed with or without added nucleating seeds. If nucleating seeds are added to the synthesis mixture, the seeds may be a SSZ-133 material obtained from a previous synthesis and may suitably present in an amount from 1 ppm by weight to 10000 ppm by weight, based on the synthesis mixture, such as from 100 ppm by weight to 5000 ppm by weight of the synthesis mixture.

In one or more aspects, the synthesis mixture after solvent adjustment (e.g., where the desired water to silica ratio is achieved) may be mixed by a mechanical process such as stirring or high shear blending to assure suitable homogenization of the base mixture, for example, using dual asymmetric centrifugal mixing with a mixing speed of 1000 rpm to 3000 rpm (e.g., 2000 rpm).

The synthesis mixture can be in the form of a solution, a colloidal dispersion (colloidal sol), gel, or paste, with a gel being preferred.

The synthesis mixture is then subject to crystallization conditions suitable for the zeolite to form. Crystallization of the zeolite may be carried out under static or stirred conditions in a suitable reactor vessel, such as for example Teflon® lined or stainless-steel autoclaves placed in a convection oven maintained at an appropriate temperature.

The crystallization in step (b) of the method is typically carried out at a temperature of 50° C. to 150° C., such as 75° C. to 125° C., for a time sufficient for crystallization to occur at the temperature used. For instance, at higher temperatures, the crystallization time may be reduced. For instance, the crystallization conditions in step (b) of the method may include heating for a period of 1 to 40 days, such as at least 1 or at least 7 or at least 10 days up to 40 or 30 or 21 days. Crystallization time can be established by methods known in the art such as by sampling the synthesis mixture at various times and determining the yield and x-ray crystallinity of precipitated solid.

Typically, the aluminosilicate zeolite is formed in solution and can be recovered by standard means, such as by centrifugation or filtration. The separated zeolite can also be washed, recovered by centrifugation or filtration and dried.

As a result of the crystallization process, the recovered product contains within its pore structure at least a portion of the organic template used in the synthesis. The as-synthesized aluminosilicate zeolite recovered from step (c) may thus be subjected to thermal treatment (e.g., calcination) or other treatment to remove part or all of the organic template incorporated into its pores during the synthesis. The thermal treatment may be carried out at a temperature in the range of from 300° C. to 900° C., for example 350° C. to 700° C., or 400° C. to 650° C. Particularly, the thermal treatment may be performed in a gas atmosphere having a temperature in the above-described ranges, which may be air, oxygen, nitrogen, or a mixture of two or more thereof. In one or more embodiments, thermal treatment is performed for a period in the range of 0.5 to 10 hours, for example 3 to 7 hours, or 4 to 6 hours.

The aluminosilicate zeolite may also be subjected to an ion-exchange treatment, for example, with aqueous ammonium salts, such as ammonium nitrates, ammonium chlorides, and ammonium acetates, in order to remove remaining alkali metal cations and/or alkaline earth metal cations and to replace them with protons thereby producing the acid form of the zeolite. To the extent desired, the original cations of the as-synthesized material, such as alkali metal cations, can be replaced by ion exchange with other cations. Preferred replacing cations can include hydrogen ions, hydrogen precursor ions (e.g., ammonium ions), and mixtures thereof. The ion exchange step may take place after the as-synthesized zeolite is dried. The ion-exchange step may take place either before or after a calcination step.

The aluminosilicate zeolite described herein is substantially crystalline. As used herein, the term “crystalline” refers to a crystalline solid form of a material, including, but not limited to, a single-component or multiple-component crystal form, e.g., including solvates, hydrates, and a co-crystal. Crystalline can mean having a regularly repeating and/or ordered arrangement of molecules and possessing a distinguishable crystal lattice. For example, the zeolite can have different water or solvent content. The different crystalline lattices can be identified by solid state characterization methods such as by XRD (e.g., powder XRD). Other characterization methods known to a person of ordinary skill in the relevant art can further help identify the crystalline form as well as help determine stability and solvent/water content. As used herein, the term “substantially crystalline” means a majority (greater than 50 wt. %) of the weight of a sample of a material described is crystalline and the remainder of the sample is a non-crystalline form. In one or more aspects, a substantially crystalline sample has at least 95% crystallinity (e.g., 5% of the non-crystalline form), at least 96% crystallinity (e.g., 4% of the non-crystalline form), at least 97% crystallinity (e.g., 3% of the non-crystalline form), at least 98% crystallinity (e.g., about 2% of the non-crystalline form), at least 99% crystallinity (e.g., 1% of the non-crystalline form), and 100% crystallinity (e.g., 0% of the non-crystalline form).

The aluminosilicate zeolite of the present disclosure, where part or all of the organic template has been removed, may be used as an adsorbent or as a catalyst or support for catalyst in a wide variety of hydrocarbon conversions (e.g., conversion of organic compounds to a converted product).

The aluminosilicate zeolite of the present disclosure (where part or all of the organic template is removed) may be used as an adsorbent, such as for separating at least one component from a mixture of components in the vapor or liquid phase having differential sorption characteristics with respect to the material. Therefore, at least one component can be partially or substantially totally separated from a mixture of components having differential sorption characteristics with respect to the zeolite by contacting the mixture with the zeolite to selectively sorb the one component. For instance, in a process for selectively separating one or more desired components of a feedstock from remaining components of the feedstock, the feedstock may be contacted with a sorbent that comprises the aluminosilicate zeolite of the present disclosure at effective sorption conditions, thereby forming a sorbed product and an effluent product. One or more of the desired components are recovered from either the sorbed product or the effluent product.

The aluminosilicate zeolite of the present disclosure (where part or all of the organic template is removed) may also be used as a catalyst to catalyze a wide variety of organic compound conversion processes. Examples of chemical conversion processes, which are effectively catalyzed by the molecular sieve described herein, either alone or in combination with one or more other catalytically active substances including other crystalline catalysts, include those requiring a catalyst with acid activity. Examples of organic conversion processes, which may be catalyzed by the aluminosilicate zeolite described herein include cracking, hydrocracking, isomerization, polymerization, reforming, hydrogenation, dehydrogenation, dewaxing, hydrodewaxing, adsorption, alkylation, transalkylation, dealkylation, hydrodecylization, disproportionation, oligomerization, dehydrocyclization, conversion of methanol to olefins, deNO, applications, and combinations thereof. The conversion of hydrocarbon feeds can take place in any convenient mode, for example in fluidized bed, moving bed, or fixed bed reactors depending on the types of process desired.

The aluminosilicate zeolite of the present disclosure may be formulated into product compositions by combination with other materials, such as binders and/or matrix materials that provide additional hardness to the finished product. These other materials can be inert or catalytically active materials.

For instance, it may be desirable to incorporate the aluminosilicate zeolite of the present disclosure with another material that is resistant to the temperatures and other conditions employed during use. Such materials include synthetic or naturally occurring zeolites as well as inorganic materials such as clays, silica and/or metal oxides such as alumina and mixtures thereof. The metal oxides may be either naturally occurring or in the form of gelatinous precipitates or gels including mixtures of silica and metal oxides. Use of a resistant material in conjunction with the aluminosilicate zeolite of the present disclosure, i.e., combined therewith or present during synthesis of the as-made zeolite, which crystal is active, tends to change the conversion and/or selectivity of the catalyst in certain organic conversion processes. Inactive resistant materials suitably serve as diluents to control the amount of conversion in a given process so that products can be obtained in an economic and orderly manner without employing other means for controlling the rate of reaction. These materials may be incorporated into naturally occurring clays (e.g., bentonite and kaolin) to improve the crush strength of the product under commercial operating conditions. These inactive resistant materials (i.e., clays, oxides, etc.) function as binders for the catalyst. A catalyst having good crush strength can be beneficial because in commercial use, it is desirable to prevent the catalyst from breaking down into powder-like materials.

Naturally occurring clays which may be used include the montmorillonite and kaolin family, which families include the subbentonites, and the kaolins commonly known as Dixie, McNamee, Georgia and Florida clays or others in which the main mineral constituent is halloysite, kaolinite, dickite, nacrite, or anauxite. Such clays can be used in the raw state as originally mined or after being subjected to calcination, acid treatment or chemical modification. Binders useful for compositing with the aluminosilicate zeolite of the present disclosure also include inorganic oxides selected from silica, zirconia, titania, magnesia, beryllia, alumina, yttria, gallium oxide, zinc oxide and mixtures thereof.

In addition to the foregoing materials, the aluminosilicate zeolite of the present disclosure may be composited with a porous matrix material such as silica-alumina, silica-magnesia, silica-zirconia, silica-thoria, silica-beryllia, silica-titania as well as ternary compositions such as silica-alumina-thoria, silica-alumina-zirconia, silica-alumina-magnesia and silica-magnesia-zirconia.

These binder materials are resistant to the temperatures and other conditions (e.g., mechanical attrition) which occur in various hydrocarbon separation processes. Thus, the aluminosilicate zeolite of the present disclosure may be used in the form of an extrudate with a binder. They are typically bound by forming a pill, sphere, or extrudate. The extrudate is usually formed by extruding the aluminosilicate zeolite, optionally in the presence of a binder, and drying and calcining the resulting extrudate. Further treatments such as steaming, and/or ion exchange may be carried out as required. The aluminosilicate zeolite may optionally be bound with a binder having a surface area of at least 100 m²/g, for instance at least 200 m²/g, or at least 300 m²/g.

The relative proportions of zeolite and inorganic oxide matrix may vary widely, with the aluminosilicate zeolite content ranging from about 1 to about 100 wt. % and more usually, particularly when the composite is prepared in the form of extrudates, in the range of 2 to about 95 wt. %, optionally from about 20 to about 90 wt. % of the composite.

### EXAMPLES

The various embodiments disclosed herein will be further clarified by the following examples. The examples are illustrative in nature and should not be understood to limit the embodiments disclosed herein.

The SiO₂:Al₂O₃ molar ratio (SAR) of was determined using Inductively Coupled Plasma Spectroscopy according to known techniques.

Micropore volume (V_{micro}) was determined by subjecting an ammonium-exchanged material after drying to a micropore volume analysis using N₂ as adsorbate and via the t-plot method, to provide a micropore volume in cm³/g. See, e.g., B. C. Lippens and J. H. de Boer, “Studies on pore systems in catalysts: V. The/method” J. Catal. 1965, 4, 319-323.

Brønsted acid site density was determined by the temperature-programmed desorption of n-propylamine. See, e.g., W. E. Farneth and R. J. Gorte. “Methods for Characterizing Zeolite Acidity”, Chem. Rev. 1995, 95, 615-635; and WO 2016/069073 A1.

### Example 1 (Comparative)

### Synthesis of PST-2 Zeolite

PST-2 was prepared in accordance with Example 1 of Korean Patent No. 10-2546689. The synthesis mixture had the following composition, in terms of molar ratios:

$$8. TEAOH : 0.5 {CsNO}_{3} : 8. {SiO}_{2} : 0.5 {Al}_{2} {O}_{3} : 240 {H}_{2} O .$$

The source of silica was colloidal silica and the source of alumina was aluminum tri-sec-butoxide.

Crystals of PST-2 display a disk-like morphology with a diameter of 1000 to 2500 nm and a thickness of 20 to 40 nm.

The as-synthesized product had an aluminum content of 6.80 wt. % (SAR=5.51).

### Example 2

### Synthesis of SSZ-133

2.54 g of aluminum hydroxide (Reheis F2000, 50% aluminum hydroxide), 9.11 g of tetramethylammonium hydroxide (TMAOH) solution, 31.55 g of tetraethylammonium hydroxide (TEAOH) solution, 6.01 g of fumed silica (available from Cabot Corporation as CAB-O-SIL® M5), and 25.37 g of deionized water were mixed together in a Teflon liner. The mixture the following composition, in terms of molar ratios:

$$2. TMAOH : 6. TEAOH : 0.5 {CsNO}_{3} : 8. {SiO}_{2} : 0.5 {Al}_{2} {O}_{3} : 240 {H}_{2} O .$$

The resulting gel was stirred until it became homogeneous and aged at 95° C. overnight. After cooling down the gel to room temperature, 1.2305 g of cesium nitrate (CsNO₃) was added and stirred for another night at room temperature. The liner was then capped, sealed inside a 125-mL autoclave, and heated at 100° C. under tumbling conditions (43 rpm) inside a convection oven for 14-21 days. The solids were then isolated by centrifugation, washed with deionized water, and dried in an oven at 95° C.

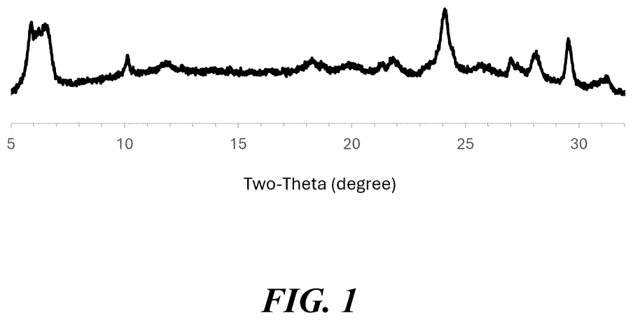

FIG. 1 shows the powder XRD pattern of the as-synthesized product and is consistent with the product being an SBS/SBT zeolite intergrowth. The powder XRD pattern of the product appears to show broad features characteristic of materials with very small crystals. This new product was identified as SSZ-133.

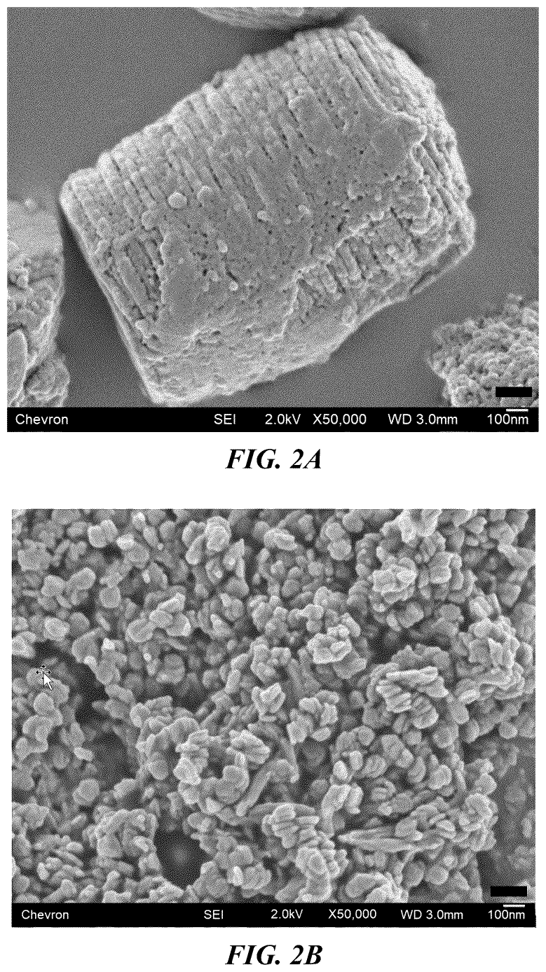

SEM images of the PST-2 zeolite of Example 1 and the as-synthesized zeolite product of Example 2 are shown in FIGS. 2A and 2B, respectively.

The as-synthesized zeolite product had a disk-like morphology, with a d50 crystal diameter of less than 50 nm and a d90 crystal diameter of less than 80 nm.

### Example 3

### Synthesis of SSZ-133

2.54 g of aluminum hydroxide (Reheis F2000, 50% aluminum hydroxide), 42.07 g of tetraethylammonium hydroxide (TEAOH) solution, 6.01 g of fumed silica (CAB-O-SIL® M5), and 25.37 g of deionized water were mixed together in a Teflon liner. The resulting gel was stirred until it became homogeneous and aged at 95° C. overnight. After cooling down the gel to room temperature, 1.2305 g of cesium nitrate (CsNO₃) was added and stirred for another night at room temperature. The mixture had the following composition, in terms of molar ratios:

$$8. TEAOH : 0.5 {CsNO}_{3} : 8. {SiO}_{2} : 0.5 {Al}_{2} {O}_{3} : 240 {H}_{2} O .$$

The liner was then capped, sealed inside a 125-mL autoclave, and heated at 100° C. under tumbling conditions (43 rpm) inside a convection oven for 14-21 days. The solids were then isolated by centrifugation, washed with deionized water, and dried in an oven at 95° C.

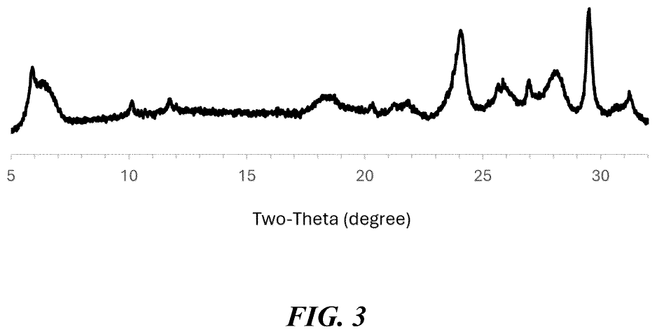

FIG. 3 shows the powder XRD of as-synthesized product and is consistent with the product being SSZ-133. The powder XRD pattern of the product appears to show broad features characteristic of materials with very small crystals.

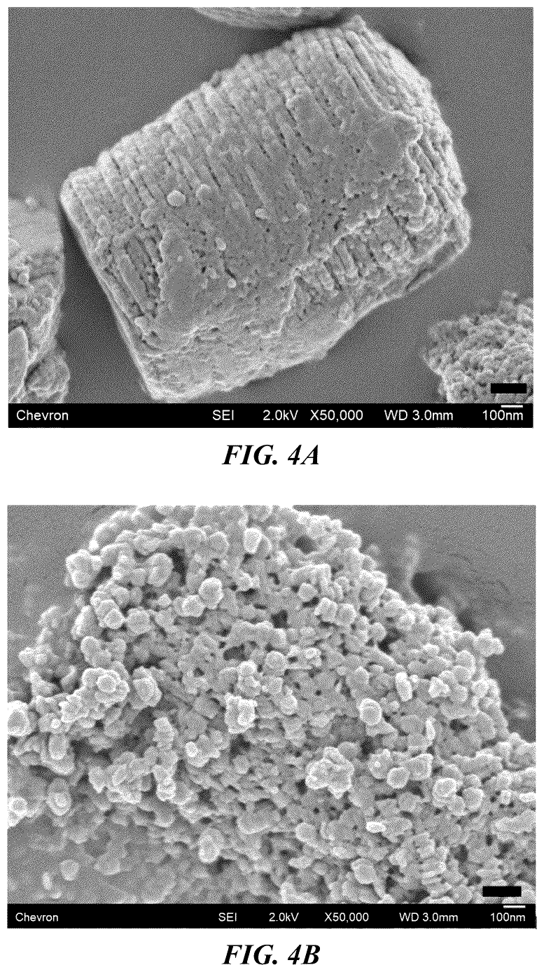

SEM images of the PST-2 zeolite of Example 1 and the as-synthesized product are shown in FIGS. 4A and 4B, respectively.

The resulting as-synthesized zeolite product had a granular or pebble morphology, with a d50 crystal diameter of less than 50 nm and a d90 crystal diameter of less than 80 nm.

### Example 4

### Synthesis of SSZ-133

Example 3 was repeated, but with a lower amount of aluminum hydroxide (1.27 g). The synthesis mixture had the following composition, in terms of molar ratios:

$$8. TEAOH : 0.5 {CsNO}_{3} : 8. {SiO}_{2} : 0.25 {Al}_{2} {O}_{3} : 240 {H}_{2} O .$$

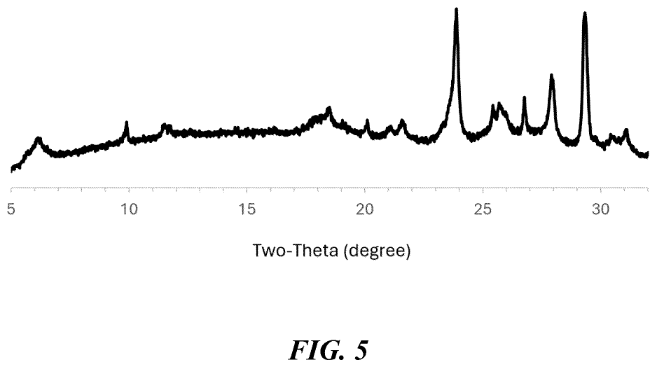

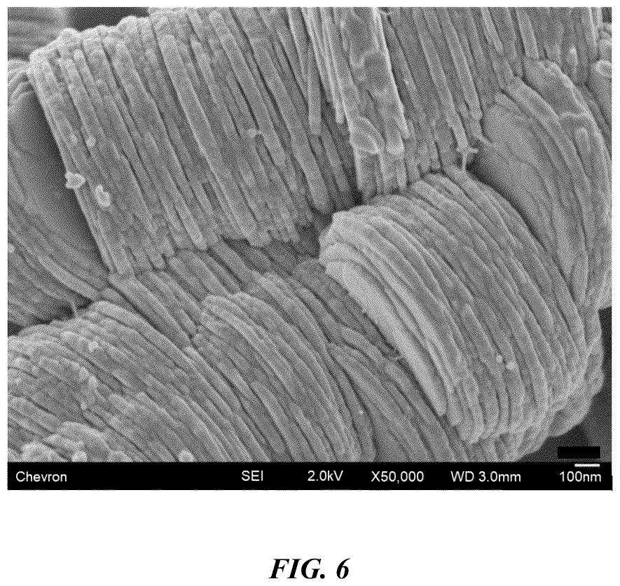

The resulting product was analyzed by powder XRD and SEM and shown to be SSZ-133, as shown in FIG. 5 and FIG. 6, respectively.

The resulting as-synthesized zeolite product had a disk-like morphology, with a d50 crystal diameter of less than 1000 nm (a d90 crystal diameter of less than 1200 μm) and a d50 crystal thickness of less than 25 nm (a d90 crystal diameter of less than 50 nm).

### Example 5

### Synthesis of SSZ-133

0.63 g of aluminum hydroxide (Reheis F2000, 50% aluminum hydroxide), 42.07 g of tetraethylammonium hydroxide (TEAOH) solution, 6.01 g of fumed silica (CAB-O-SIL® M5), and 26.33 g of deionized water were mixed together in a Teflon liner. The resulting gel was stirred until it became homogeneous and aged at 95° C. overnight. After cooling down the gel to room temperature, 0.3076 g of cesium nitrate (CsNO₃) was added and stirred for another night at room temperature. The mixture had the following composition, in terms of molar ratios:

$$8. TEAOH : 0.125 {CsNO}_{3} : 8. {SiO}_{2} : 0.125 {Al}_{2} {O}_{3} : 240 {H}_{2} O .$$

The liner was then capped, sealed inside a 125-mL autoclave, and heated at 100° C. under tumbling conditions (43 rpm) inside a convection oven for 14-21 days. The solids were then isolated by centrifugation, washed with deionized water, and dried in an oven at 95° C.

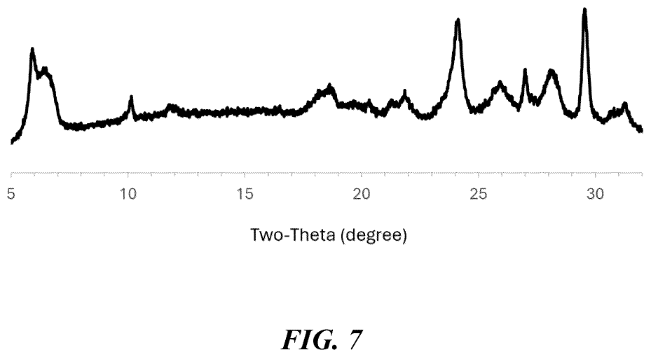

FIG. 7 shows the powder XRD of the as-synthesized product and is consistent with the product being a SSZ-133. The powder XRD pattern of the product appears to show broad features characteristic of materials with very small crystals.

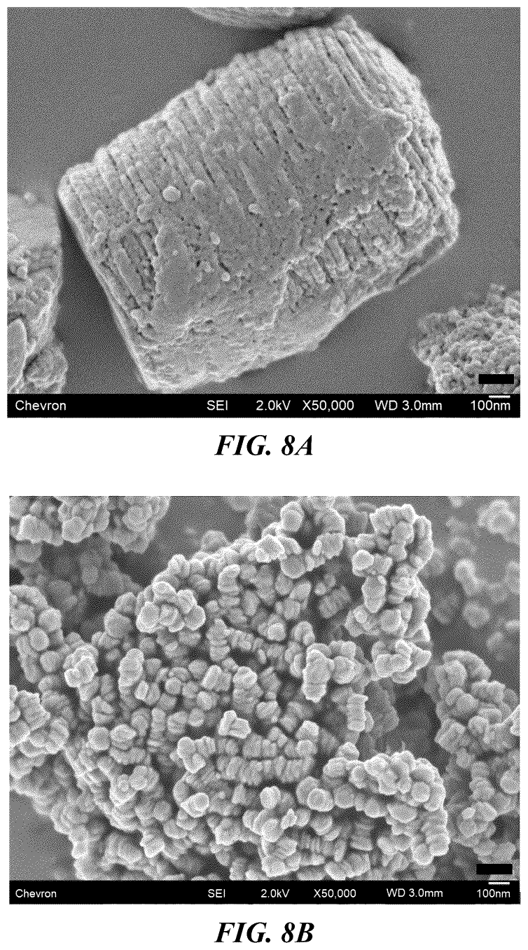

SEM images of the PST-2 zeolite of Example 1 and the as-synthesized zeolite product are shown in FIGS. 8A and 8B, respectively.

The resulting as-synthesized zeolite product had a granular or pebble morphology, with a d50 crystal diameter of less than 50 nm and a d90 crystal diameter of less than 80 nm.

### Example 6

### Synthesis of SSZ-133

Example 5 was repeated, but with a larger amount of cesium nitrate (0.61 g). The mixture had the following composition, in terms of molar ratios:

$$8. TEAOH : 0.25 {CsNO}_{3} : 8. {SiO}_{2} : 0.125 {Al}_{2} {O}_{3} : 240 {H}_{2} O .$$

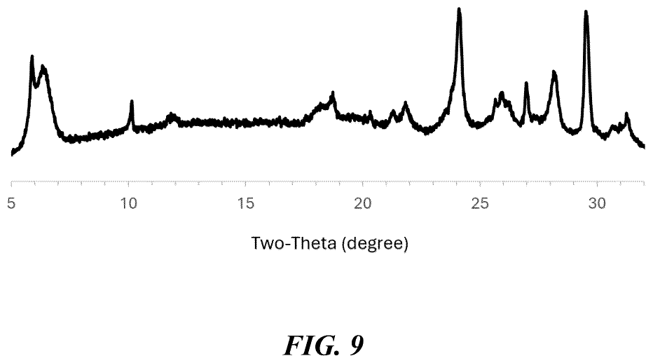

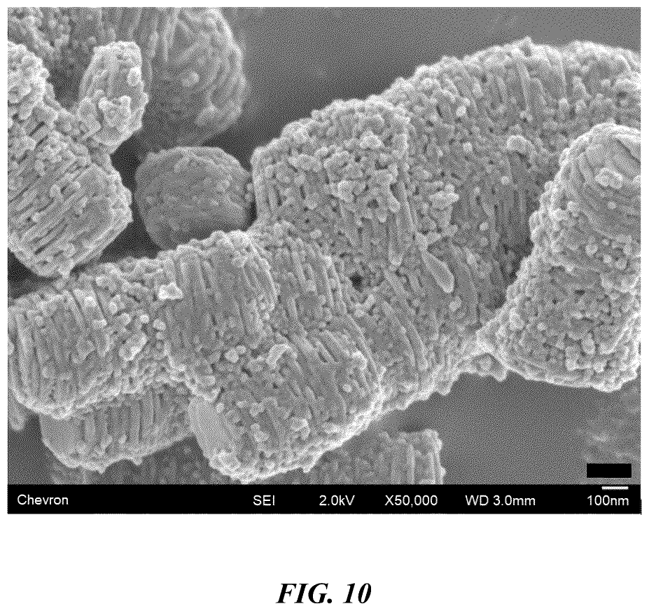

The resulting product was analyzed by powder XRD and SEM and shown to be SSZ-133, as shown in FIG. 9 and FIG. 10, respectively.

The resulting as-synthesized zeolite product had a disk-like morphology, with a d50 crystal diameter of less than 200 nm (a d90 crystal diameter of less than 400 nm) and a d50 crystal thickness of less than 12 nm (a d90 crystal thickness of less than 25 nm).

### Calcination and Ion-Exchange of Zeolites

The as-synthesized products from Examples 2-6 were converted into the sodium form under an atmosphere of dry air at a heating rate of 1° C./min to 120° C. and held for 2 hours followed by a second ramp of 1° C./min to 540° C. and held at this temperature for 3 hours and lastly a third ramp of 1° C./min to 595° C. and held at this temperature for 5 hours. Finally, the sample was cooled down to 120° C. or below. Each of these calcined samples was then exchanged into the ammonium form as follows. An amount of ammonium nitrate equal to the mass of the sample to be exchanged was fully dissolved in an amount of deionized water ten times the mass of the sample. The sample was then added to the ammonium nitrate solution and the suspension was sealed in a flask and heated in an oven at 85° C. for at least 3 hours. The flask was removed from the oven, and the sample was recovered immediately by filtration. This ammonium exchange procedure was repeated on the recovered sample, washed with copious amounts of deionized water to a conductivity of less than 100 μS/cm and finally dried in an oven at 85° C. for 3 hours.

Table 1 summarizes the physical and chemical properties of the zeolite products of Examples 2-6.

TABLE 1
Zeolite Chemical and Physical Properties

| | | Brønsted Acidity^{(b)} | V_{micro}^{(b)} |
|---|---|---|---|
| | SAR^{(a)} | [mmol/g] | [cm³/g] |
| Example 2 | 5.51 | 1107 | 0.24 |
| Example 3 | 5.43 | 1221 | 0.23 |
| Example 4 | 5.84 | 1481 | 0.31 |
| Example 5 | 6.89 | 1658 | 0.37 |
| Example 6 | 7.82 | 1494 | 0.31 |

^{(a)}As-synthesized zeolite
^{(b)}Ammonium-exchanged zeolite

### Catalyst Preparation and Evaluation

Palladium ion-exchange was carried out on each of the ammonium-exchanged samples from Examples 2-6 using tetraamminepalladium (II) nitrate (0.5 wt. % Pd). After ion-exchange, the samples were dried at 85° C. and then calcined in air at 482° C. for 3 hours to convert the tetraamminepalladium (II) nitrate to palladium oxide. Finally, each Pd-exchanged catalyst was pelletized at 5 kpsi, crushed and sieved to 20-40 mesh.

The Pd-exchanged catalysts prepared were evaluated in an n-hexadecane (n-C16) model compound isomerization test as described in U.S. Pat. No. 5,282,958, hereby incorporated by reference.

0.5 g of each of the palladium-exchanged samples from Examples 2-6 was loaded in the center of a 23 inch-long by 0.25 inch outside diameter stainless-steel reactor tube with alundum loaded upstream of the catalyst for pre-heating the feed (total pressure of 1200 psig; down-flow hydrogen rate of 160 mL/min, when measured at 1 atmosphere pressure and 25° C.; down-flow liquid feed rate of 1 mL/hour). All materials were first reduced in flowing hydrogen at about 315° C. for 1 hour. Products were analyzed by on-line capillary gas chromatography (GC) once every thirty minutes. Raw data from the GC was collected by an automated data collection/processing system and hydrocarbon conversions were calculated from the raw data.

The catalyst was tested at about 260° C. initially to determine the temperature range for the next set of measurements. The overall temperature range will provide a wide range of n-hexadecane conversion with the maximum conversion just below and greater than 96%. At least five on-line GC injections were collected at each temperature. Conversion was defined as the amount of n-hexadecane (n-C16) reacted to produce other products (including iso-C16 isomers). Yields were expressed as weight percent of products other than n-C16 and included iso-C16 as a yield product.

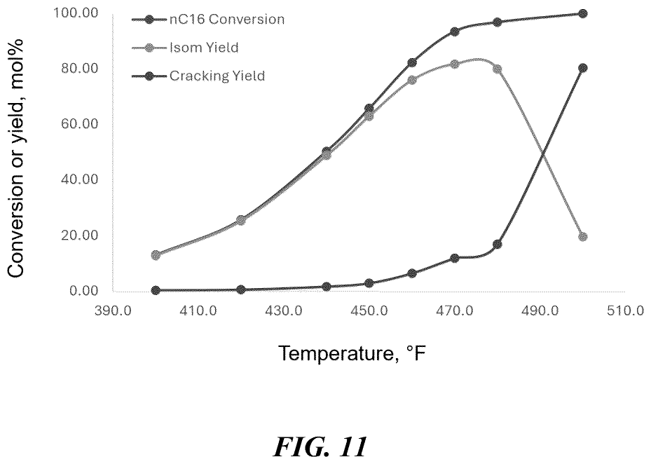

FIG. 11 shows a plot of conversion or yield versus temperature for n-C16 conversion over a Pd/SSZ-133 catalyst prepared using the SSZ-133 zeolite of Example 2.

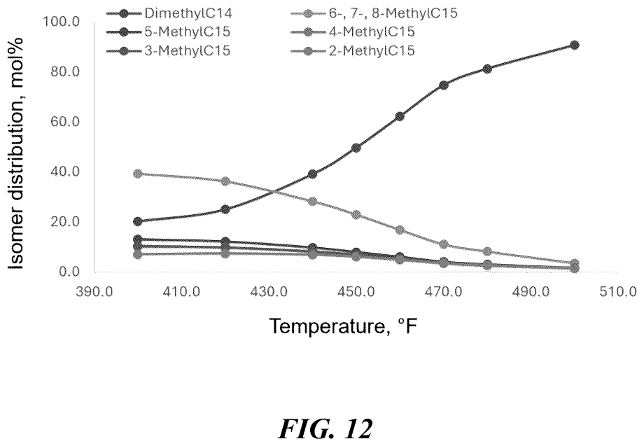

FIG. 12 shows a plot of isomer distribution versus temperature for n-C16 conversion over the Pd/SSZ-133 catalyst prepared using the SSZ-133 zeolite of Example 2.

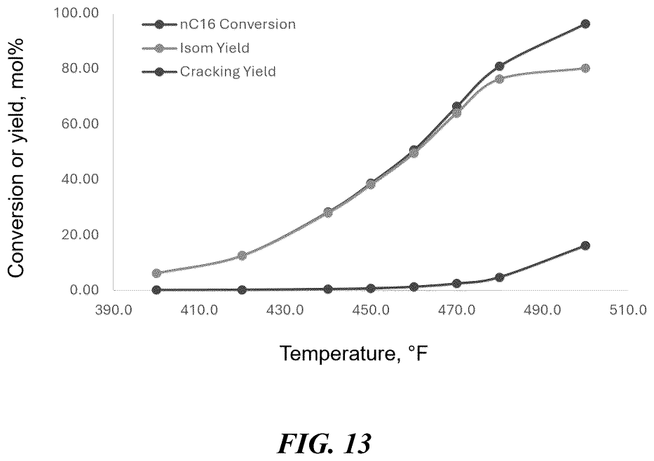

FIG. 13 shows a plot of conversion or yield versus temperature for n-C16 conversion over a Pd/SSZ-133 catalyst prepared using the SSZ-133 zeolite of Example 3.

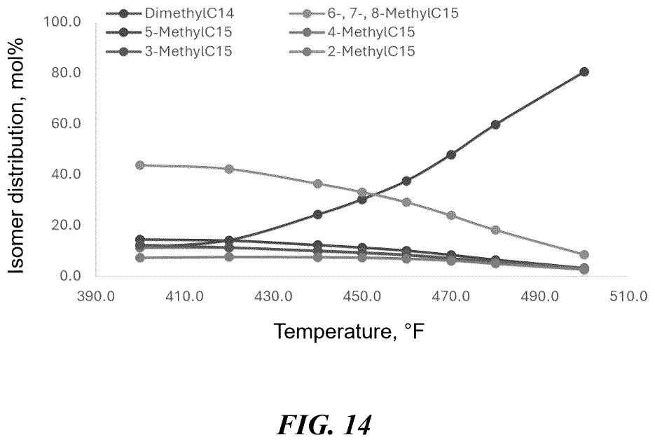

FIG. 14 shows a plot of isomer distribution versus temperature for n-C16 conversion over the Pd/SSZ-133 catalyst prepared using the SSZ-133 zeolite of Example 3.

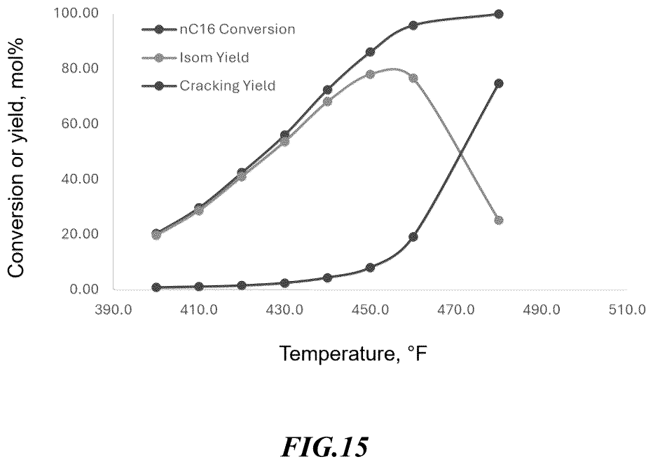

FIG. 15 shows a plot of conversion or yield versus temperature for n-C16 conversion over a Pd/SSZ-133 catalyst prepared using the SSZ-133 zeolite of Example 4.

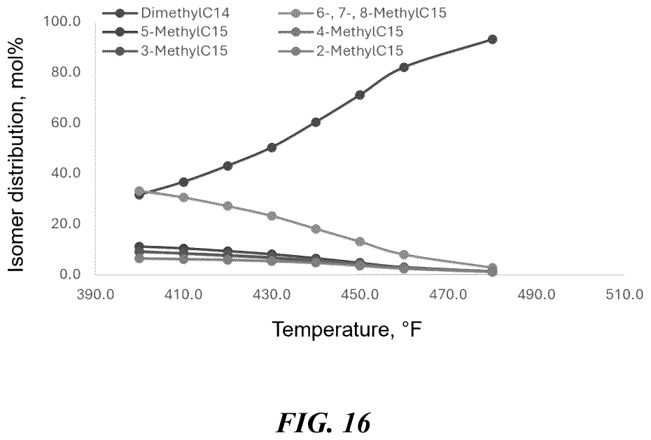

FIG. 16 shows a plot of isomer distribution versus temperature for n-C16 conversion over the Pd/SSZ-133 catalyst prepared using the SSZ-133 zeolite of Example 4.

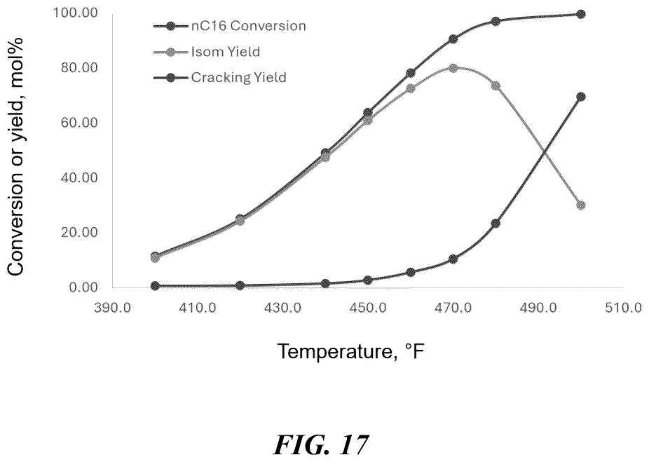

FIG. 17 shows a plot of conversion or yield versus temperature for n-C16 conversion over a Pd/SSZ-133 catalyst prepared using the SSZ-133 zeolite of Example 5.

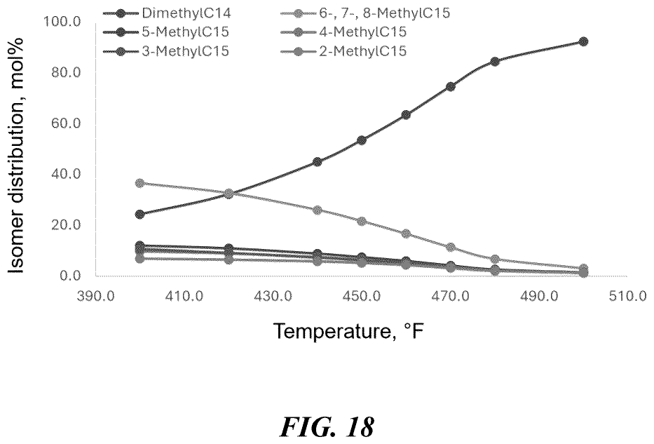

FIG. 18 shows a plot of isomer distribution versus temperature for n-C16 conversion over the Pd/SSZ-133 catalyst prepared using the SSZ-133 zeolite of Example 5.

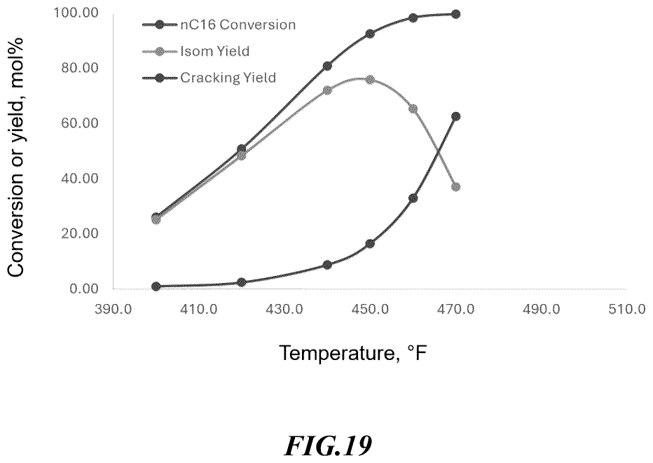

FIG. 19 shows a plot of conversion or yield versus temperature for n-C16 conversion over a Pd/SSZ-133 catalyst prepared using the SSZ-133 zeolite of Example 6.

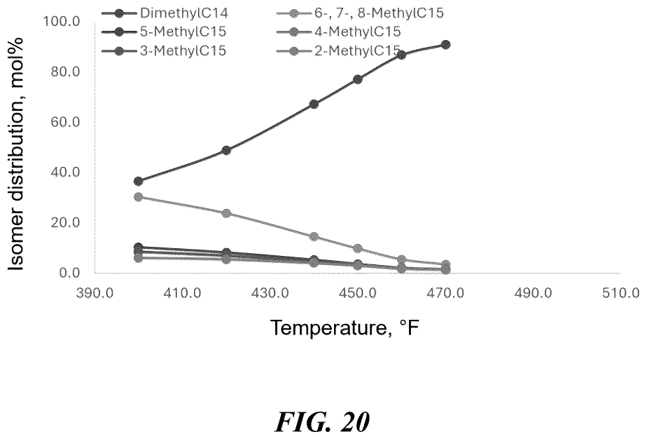

FIG. 20 shows a plot of isomer distribution versus temperature for n-C16 conversion over the Pd/SSZ-133 catalyst prepared using the SSZ-133 zeolite of Example 6.

What is claimed is:

1. An aluminosilicate zeolite having an intergrowth of a SBS framework structure and a SBT framework structure, wherein the zeolite has a d50 crystal size of 500 μm or less.

2. The zeolite of claim 1, wherein the zeolite has a d90 crystal size of 500 μm or less.

3. The zeolite of claim 1, wherein the zeolite has a SiO₂/Al₂O₃ molar ratio of 1 to 65.

4. The zeolite of claim 1, wherein the zeolite has a SiO₂/Al₂O₃ molar ratio of 5 to 10.

5. The zeolite of claim 1, comprising a tetraethylammonium cation or a combination of a tetraethylammonium cation and a tetramethylammonium cation within its pore structure.

6. A method of making the zeolite of claim 1, the method comprising:
(a) preparing a synthesis mixture comprising: a source of alumina, a source of silica, an organic template (Q) comprising a tetraethylammonium cation, a source of hydroxide ions (OH), a source of cesium cation (Cs), and water;
(b) heating the synthesis mixture under crystallization conditions including a temperature of from 50° C. to 150° C. for a time sufficient to form crystals of the zeolite; and
(c) recovering at least a portion of the zeolite from step (b);
wherein the source of alumina is free of, essentially free of, or substantially free of an aluminum alkoxide and wherein the source of silica is free of, essentially free of, or substantially free of colloidal silica.

7. The method of claim 6, wherein the synthesis mixture has the following composition in terms of molar ratios:

| | |
|---|---|
| SiO₂/Al₂O₃ | 5 to 70 |
| Q/SiO₂ | 0.5 to 1.5 |
| OH/SiO₂ | 0.75 to 1.75 |
| Cs/SiO₂ | 0.01 to 0.15 |
| H₂O/SiO₂ | 10 to 60. |

8. The method of claim 6, wherein the organic template (Q) is in the form of a hydroxide.

9. The method of claim 6, wherein the source of alumina consists of or consists essentially of aluminum hydroxide.

10. The method of claim 6, wherein the source of silica consists of or consists essentially of fumed silica.

11. The method of claim 6, wherein the organic template further comprises a tetramethylammonium cation.

12. The method of claim 11, wherein a molar ratio of the tetraethylammonium cation to the tetramethylammonium cation is from 1:1 to 10:1.

13. The method of claim 1, further comprising (d) treating the zeolite recovered in step (c) to remove at least part of the organic template (Q).

14. A process of converting an organic compound to a conversion product comprises contacting the organic compound with the zeolite of claim 1.
